(19) United States
(12) Patent Application Publication
(10) Pub. No.: US 2026/0020676 A1
(43) Pub. Date: Jan. 22, 2026

(54) SELF-STORING MULTIFUNCTIONAL TRAVEL SET

(71) Applicants: Bonnie Harriet Hartstein, San Antonio, TX (US); Gal Shweiki, San Antonio, TX (US)

(72) Inventors: Bonnie Harriet Hartstein, San Antonio, TX (US); Gal Shweiki, San Antonio, TX (US)

(21) Appl. No.: 19/275,498

(22) Filed: Jul. 21, 2025

Related U.S. Application Data
(60) Provisional application No. 63/673,755, filed on Jul. 21, 2024.

(51) Int. Cl. A47C 7/38; A47G 9/02; A61F 9/04

(52) U.S. Cl. CPC A47C 7/383; A47G 9/0223; A61F 9/04

(57) ABSTRACT

A self-storing multifunctional travel set includes a compressible headrest pillow, a removable eye mask, and a sling-style blanket integrated into a compact, portable unit. The headrest pillow defines an internal storage cavity accessible via a zippered closure, dimensioned to enclose the eye mask and sling-style blanket when not in use. The set is configurable between a deployed state, in which the components provide head, eye, and torso support during seated travel, and a stored state, in which all components are enclosed within the pillow. One or more straps are affixed to the pillow and serve both to compress the stored contents and to secure the pillow to a seatback during use. The sling-style blanket includes anchoring elements and adjustable connectors for stabilizing the torso, while the eye mask includes a strap and is compactable for storage. All components are fabricated from washable, compressible materials suitable for portable travel use.

### CROSS-REFERENCE TO RELATED APPLICATIONS

The present U.S. Utility Patent application claims priority pursuant to 35 U.S.C. § 119 (e) to U.S. Provisional Application No. 63/673,755, filed on Jul. 21, 2024, the entire contents of which are hereby incorporated by reference in its entirety and made part of the present U.S. Utility Patent Application for all purposes.

### STATEMENT REGARDING FEDERALLY SPONSORED RESEARCH DEVELOPMENT

Not applicable.

### TECHNICAL FIELD

The disclosed embodiments relate to the field of travel accessories, more specifically to portable self-storing multifunctional travel sets that combine a pillow, an eye mask, and a sling-style blanket, designed to enhance comfort and sleep quality while seated during travel, and improve portability.

### BACKGROUND

Travel, particularly over long distances, often results in physical discomfort due to inadequate neck and back support, ambient light disruption, and a lack of warmth. While accessories such as neck pillows, eye masks, and travel blankets are commonly used to address these issues, they are typically offered as separate items or bundled in a non-integrated fashion, often relying on a separate external pouch for storage. These separate pouches are frequently lost or misplaced during transit, resulting in disorganization and user frustration.

Moreover, many of these accessories must be folded or packed in a specific way to fit back into their carrying case, which is not always intuitive or convenient, especially in tight travel conditions. Improper repacking leads to bulky and awkward storage, resulting in a diminished user experience. Additionally, current travel sets often fail to promote deep, restorative sleep or proper ergonomic support. Users who fall asleep without adequate neck and torso alignment may experience pain, stiffness, or longer-term posture issues upon waking.

There exists a need for an integrated travel system that not only consolidates multiple comfort functions but also simplifies storage and enhances usability.

### SUMMARY

The present invention addresses the shortcomings of traditional travel accessories by introducing a self-storing, multifunctional travel set that integrates a headrest pillow, an eye mask, and a sling-style blanket into a compact, ergonomic unit. Unlike existing travel sets that rely on separate pouches or precise folding techniques, this invention consolidates essential elements of travel comfort-neck support, body stabilization, and light isolation-into a single, portable solution designed to support restorative upright sleep.

The system comprises a contoured headrest pillow configured to cradle the user's head and maintain cervical alignment; an adjustable eye mask for blocking ambient light, which can be used independently or attached to the pillow for enhanced head support; and a soft, sling-style blanket that wraps around the torso and arms to provide warmth and minimize body movement. When the eye mask is affixed to the pillow, the system wraps around the user's head, anchoring it in place and mitigating the common issue of head drop during seated rest. When used in conjunction with the torso-stabilizing sling-style blanket, the complete system promotes upright posture and reduces muscular strain, offering a comprehensive solution for achieving deep, restorative rest while in transit.

After use, the eye mask and sling-style blanket can be manually folded and stored within an internal storage cavity of the pillow. One or more adjustable straps, permanently affixed within the cavity, are used to compress and secure the stored components, allowing the unit to maintain a compact shape even if the items are loosely packed. These same straps also serve to fasten the pillow to a seatback or headrest during use, ensuring consistent positioning and minimizing slippage.

All components are constructed from durable, breathable materials suitable for use across the pillow, eye mask, and sling-style blanket, such as cotton, nylon, or modal. Internal components of the pillow and eye mask may include memory foam, polyurethane foam, or polyester fiberfill to provide structure, cushioning, and ergonomic support. The invention eliminates the need for external carrying cases, reduces the risk of item loss, and simplifies the packing process. When fully packed, the system fits easily within standard backpacks, laptop bags, or carry-on luggage, or can be externally secured using its integrated hook, making it an ideal solution for travelers seeking enhanced organization, portability, sleep quality, and comfort while seated.

Additional aspects of the disclosed embodiment will be outlined in part in the description that follows or will be evident from it or learned by practicing the invention. The aspects of the disclosed embodiments will be attained by means of the elements and combinations particularly pointed out in the appended claims. It is to be understood that both the foregoing general description and the following detailed description are exemplary and explanatory only and are not restrictive of the disclosed embodiments, as claimed.

### BRIEF DESCRIPTION OF THE DRAWINGS

The accompanying drawings, which are incorporated in and constitute part of this specification, illustrate embodiments of the claimed subject matter and, together with the description, explain the disclosed embodiments' principles. The embodiments illustrated herein are presently preferred, it being understood, however, that the claimed subject matter is not limited to the precise arrangements and instrumentalities shown, wherein:

FIG. 1 is a perspective view of the self-storing multifunctional travel set in its stored position.

FIG. 2 is a perspective view of the self-storing multifunctional travel set in its open position, showing the internal storage cavity of the pillow with the securing straps.

FIG. 3 is a top view thereof.

FIG. 4 is a bottom view thereof, showing the full pillow configuration.

FIG. 5 is a perspective view of the eye mask with the strap deployed for separate use.

FIG. 6 is a front view thereof.

FIG. 7 is a rear view thereof, showing the internal additional padding.

FIG. 8 is a left side view thereof, showing the strap storage pocket.

FIG. 9 is a right side view thereof, showing the securing hook engaged with the securing end.

FIG. 10 is a perspective view of the sling-style blanket in an extended configuration.

FIG. 11 is a side view of the self-storing multifunctional travel set with the eye mask and pillow configuration arranged for use.

FIG. 12 is a front top view thereof.

FIG. 13 is a rear top view thereof.

FIG. 14 is a perspective view of the self-storing multifunctional travel set in its open position with the eye mask and sling-style blanket securely stored inside.

FIG. 15 is a perspective view of the self-storing multifunctional travel set in use within a travel environment.

### DETAILED DESCRIPTION

The following detailed description refers to the accompanying drawings, with consistent reference numbers used for corresponding elements. While certain embodiments are described, modifications and alternatives are possible. Elements may be substituted, added, or modified, and method steps may be reordered or adjusted. Accordingly, this description does not limit the invention; the scope is defined solely by the appended claims.

The present invention improves upon traditional travel sets by providing a self-contained, integrated system comprising a headrest pillow, an eye mask, and a sling-style blanket, all designed to work together to provide ergonomic stability, warmth, and ambient light isolation during upright sleep while traveling. In the preferred embodiment, these components function as a unified, self-storing system that supports the user's neck and torso while promoting restful upright sleep, eliminating the need for a separate carrying pouch, and allowing for intuitive packing and unpacking.

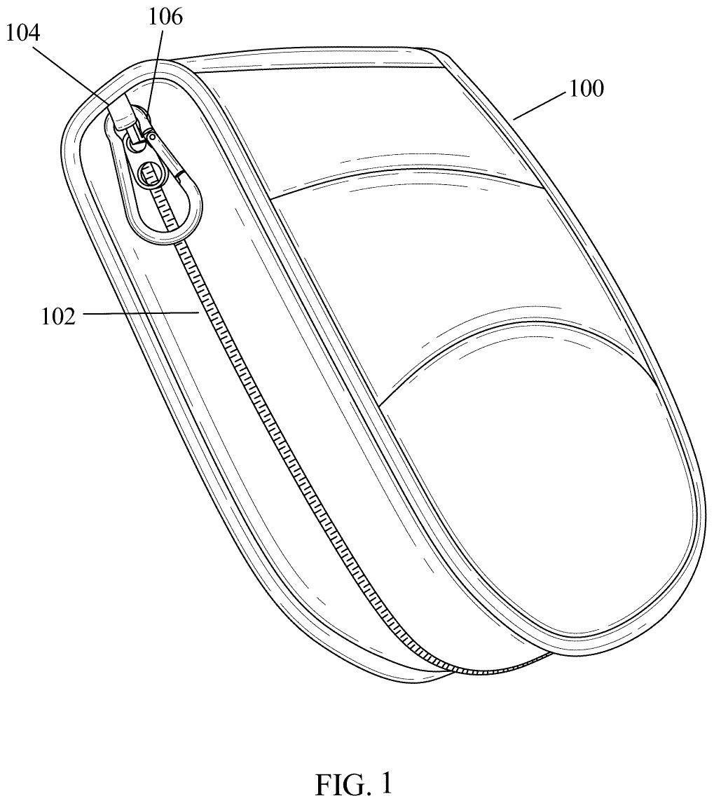

Referring now to FIG. 1, a perspective view of the self-storing multifunctional travel set in its fully stored configuration is shown. The external structure features a soft, contoured pillow 100, which is equipped with a closable zipper 102 that provides access to an internal pillow storage cavity 200. Affixed to the pillow is a reinforced carry loop 104 with a hook 106 attached to it, which allows the device to be transported or externally mounted to a backpack or luggage.

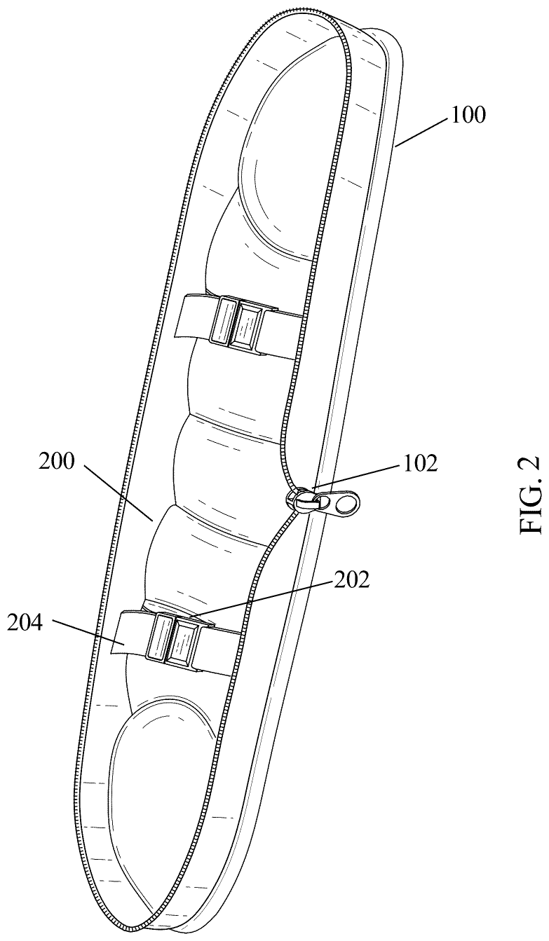

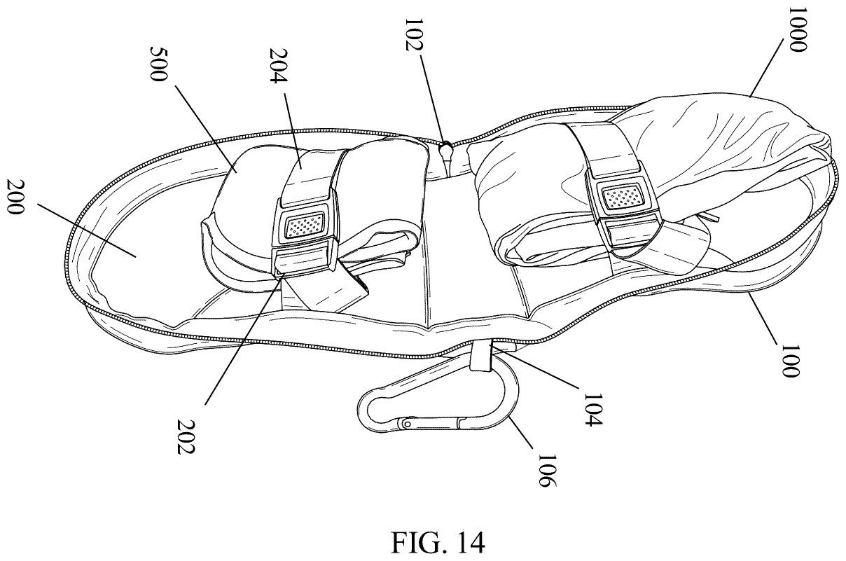

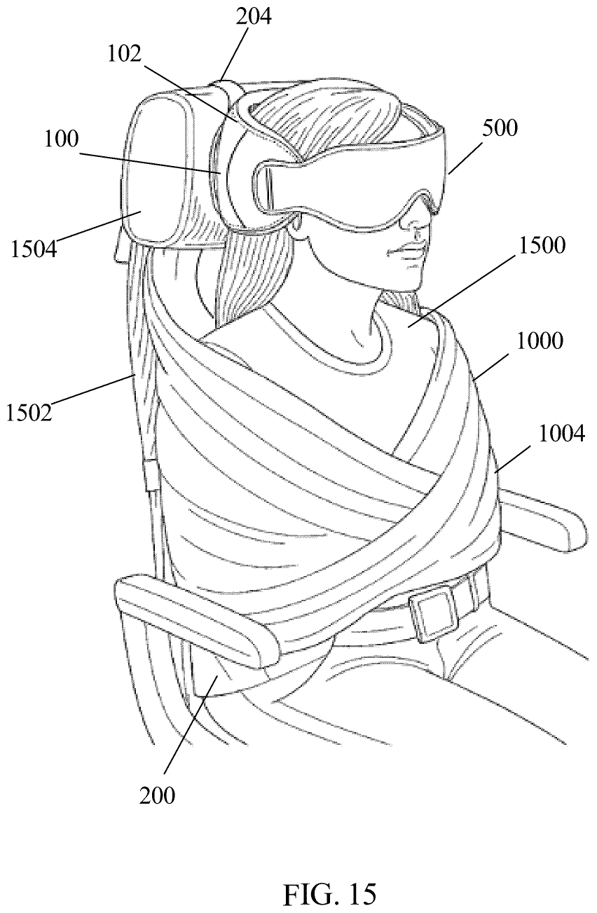

Turning to FIG. 2, the pillow 100 is shown in an open configuration, where the zipper 102 is disengaged, revealing the internal pillow storage cavity 200. Inside the cavity are securing straps 204, each terminating in buckle elements 202. These straps serve a dual function: they may be used to compress and secure the eye mask 500 and sling-style blanket 1000 within the storage cavity 200 during transport, as shown in FIG. 14, and also as a fastening mechanism to secure the pillow 100 to a seat 1502 during use, as illustrated in FIG. 15.

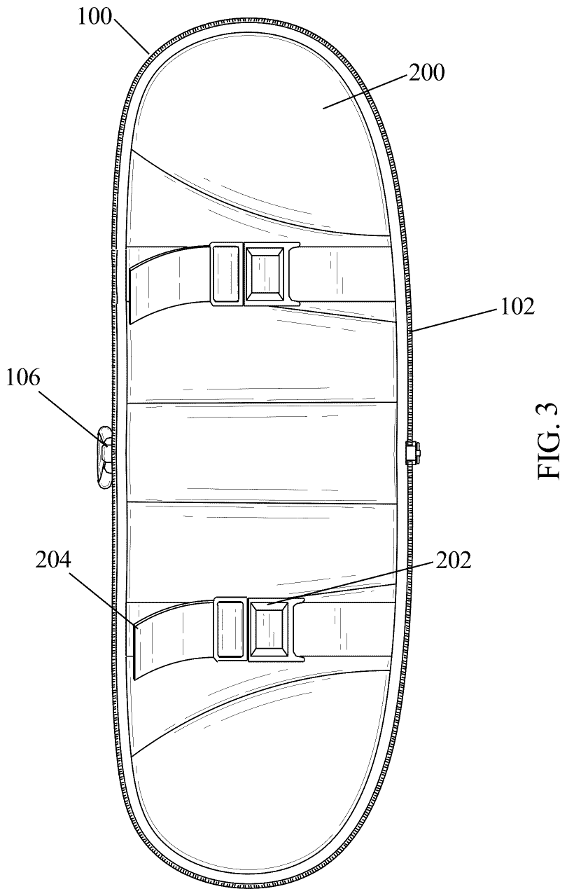

As shown in FIG. 3, a top view of the open pillow 100 reveals the spatial positioning of the zipper 102, hook 106, pillow storage cavity 200, and the buckle-equipped securing straps 204. The straps 204 are sewn into the inner walls of the cavity 200 to prevent detachment or loss.

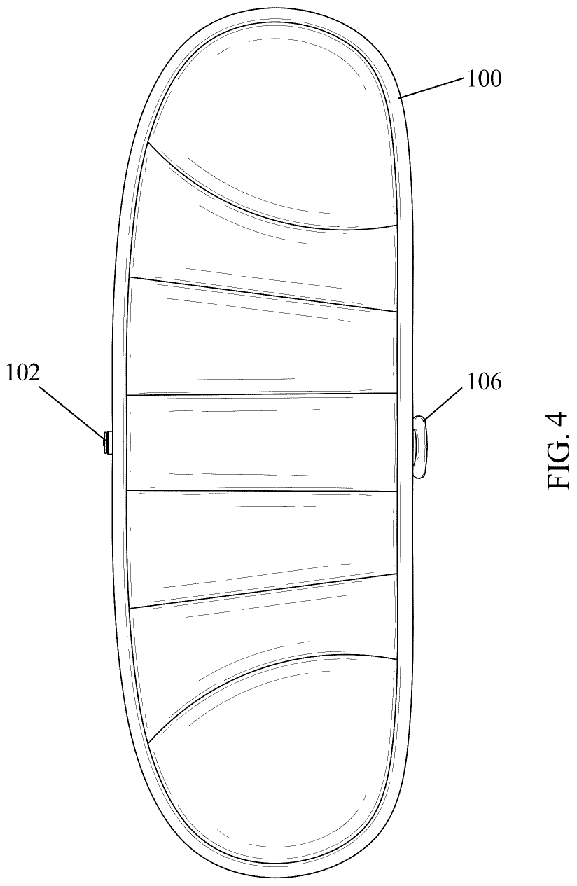

FIG. 4 shows a bottom view of the open pillow 100, depicting the overall underside of the pillow's body, which will be in direct contact with the user's 1500 head. This view further highlights that the structure is symmetrical along one axis, allowing for reversible usage.

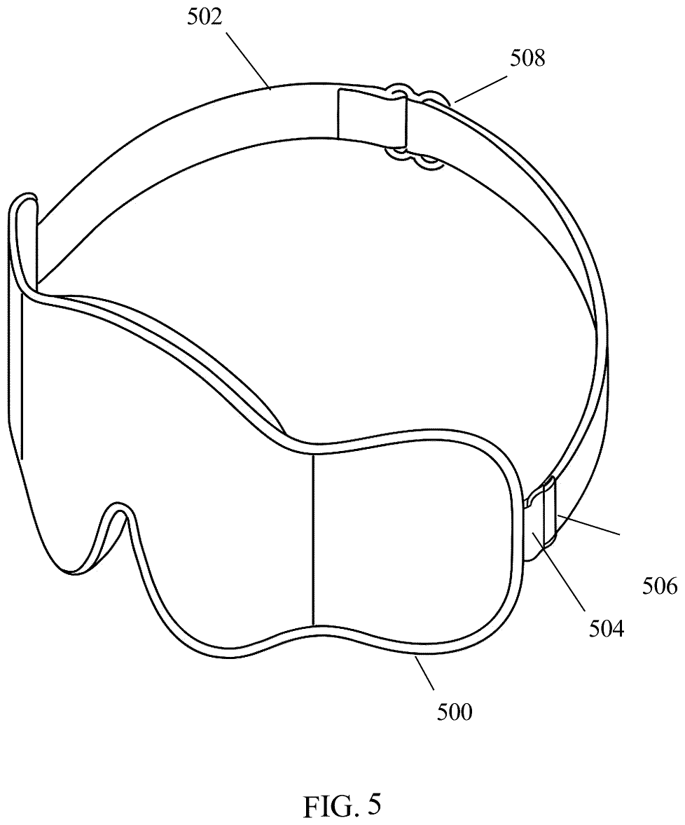

Attention is now directed to FIGS. 5 through 9, which illustrate the components and features of the eye mask assembly 500. In FIG. 5, a perspective view of the eye mask 500 is shown with a strap 502 deployed. The strap 502 includes an adjusting mechanism 508 that allows the user to customize fit and tension, and a securing hook 506 that attaches to the securing end 504 at the opposite end of the eye mask 500 for deployment. The strap 502 is permanently affixed on one end, and may be stored inside a strap storage pocket 600 located on the right side of the mask to prevent loss.

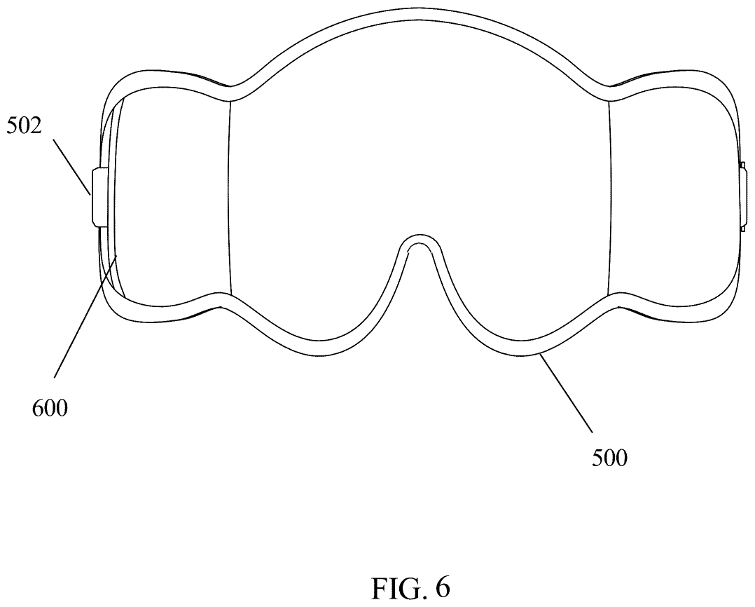

FIG. 6 provides a front view of the eye mask 500, displaying the strap 502 in its extended position and the strap storage pocket 600, which is integrated into the right portion of the mask body.

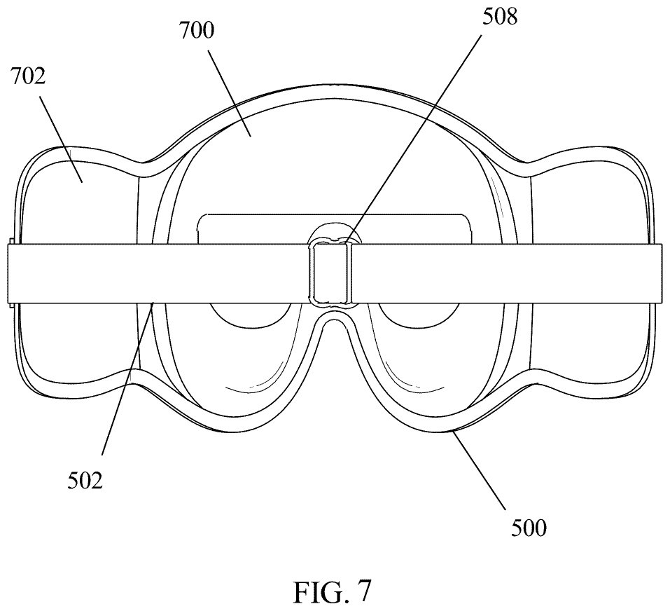

In FIG. 7, a rear view of the eye mask 500, reveals additional mask cushioning 700 applied to the surface intended to make contact with the user's face 1500. This padding enhances comfort and helps block ambient light. Also visible is an attaching medium 702, such as a Velcro or magnetic interface, used for temporarily coupling the eye mask 500 to the pillow 100.

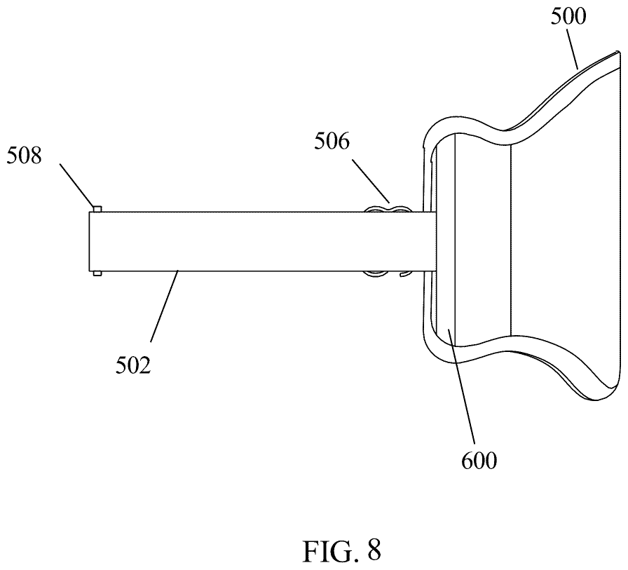

FIG. 8 is a left side view of the eye mask 500 with the deployed strap 502, highlighting the strap storage pocket 600, which can be used for the clean storage of the strap 502 when the mask 500 is used in combination with the pillow 100 or when packed into the pillow cavity 200.

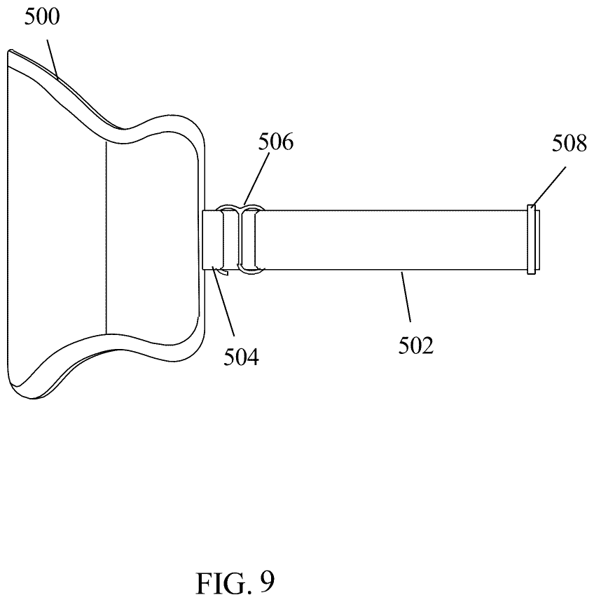

FIG. 9 shows a right side view of the eye mask 500 with the deployed strap 502, highlighting the securing end 504 coupled to the securing hook 506, as well as the adjusting mechanism 508. These features facilitate ergonomic adjustment and tension control when the mask 500 is worn separately from the pillow.

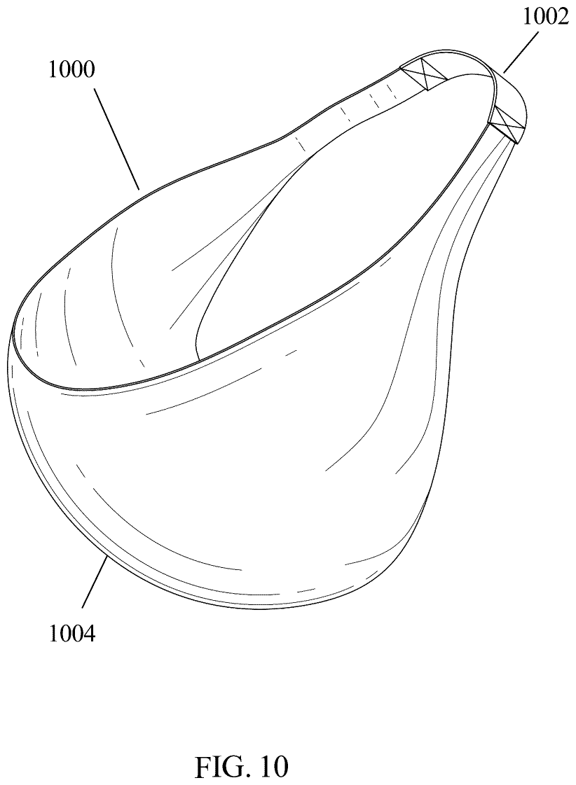

Referring now to FIG. 10, a perspective view of the sling-style blanket 1000 is presented. The sling-style blanket 1000 comprises a panel of breathable fabric 1004 and incorporates an elastic securing mechanism 1002, which allows it 1000 to be wrapped around the user's torso or secured to a seat 1502. This feature enhances torso stabilization and provides warmth without causing overheating, thereby promoting restful, upright sleep and relaxation.

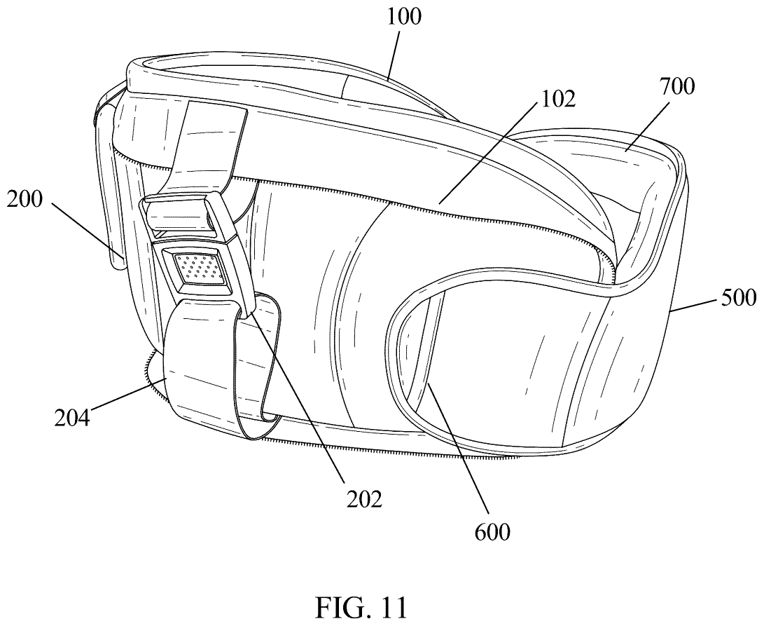

Attention is now directed to FIGS. 11 through 13, which show various configurations of the pillow 100 and eye mask 500 assembly. These figures illustrate the positional relationships between components in the deployed arrangement.

In FIG. 11, a side view of the pillow 100 in curved position is shown, with the eye mask 500 coupled via the attaching medium 702, forming a stable structural sleep aid. The securing straps 204 and buckle elements 202 in this assembly configuration will help secure the pillow 100 to the seat 1502.

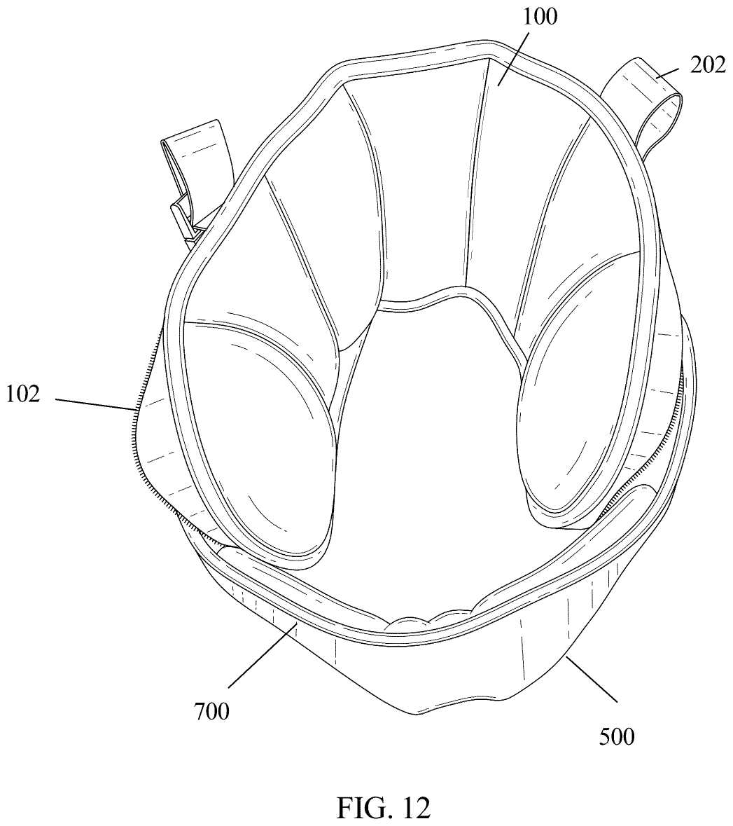

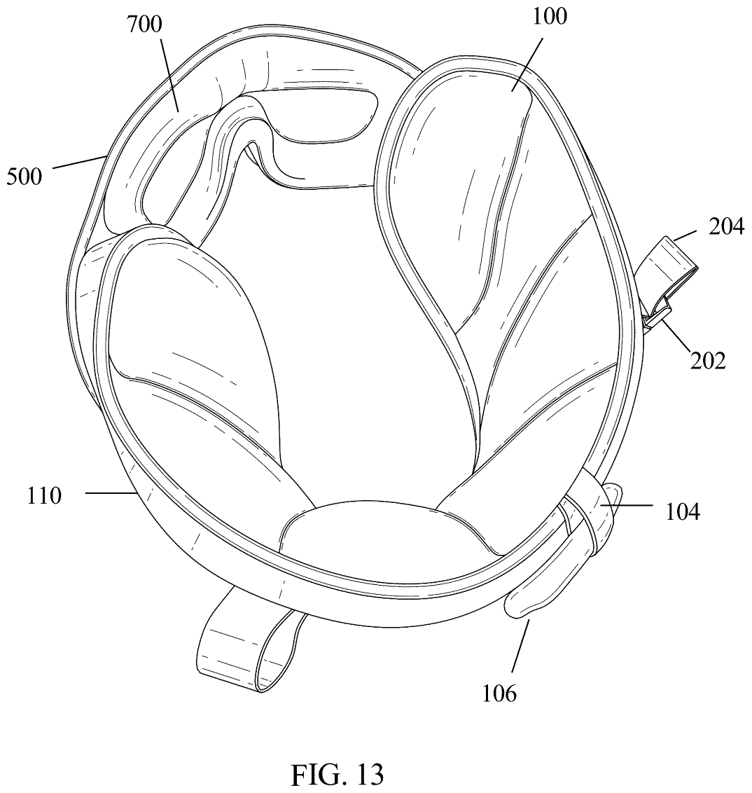

FIG. 12 and FIG. 13 are further views of the pillow 100 and eye mask 500 assembly from the top, showing how the components integrate spatially when the system is assembled for use.

Turning now to FIG. 14, a perspective view is shown of the pillow 100 in its open configuration, with both the eye mask 500 and sling-style blanket 1000 stored within the pillow storage cavity 200. The zipper 102 is open, and the securing straps 204, with their buckle elements 202, are engaged to compress the internal contents, while the zipper 102, hook 106, and carry loop 104 maintain exterior security and portability. This figure illustrates that the travel system is entirely self-contained and can be re-packed without external storage components.

Finally, FIG. 15 illustrates the self-storing multifunctional travel set in use by a user 1500 seated in a seat 1502. The pillow 100 and eye mask 500 are connected and cradle the user's head, supporting it in a stable, ergonomic position while providing full light coverage. The securing straps 204, attached to buckle elements 202, are used to fasten the pillow 100 to the seat's headrest 1504, offering cervical stability and reducing head movement. Although the elastic securing mechanism 1002 is not explicitly shown in this figure, it may be wrapped around the headrest 1504 or the user's torso to secure the sling-style blanket 1000 in place. The blanket's breathable fabric 1004 gently envelops the user's 1500 upper body, reducing torso sway and providing warmth without overheating. In this stable, upright sleep configuration, users can achieve deeper and more restorative sleep while traveling.

Accordingly, the preferred embodiment of the self-storing multifunctional travel set integrates the functional benefits of a neck pillow, eye mask, and sling-style blanket into a compact, deployable, and self-contained system. Each component physically and functionally interacts to support upright posture, reduce the risk of item loss, enhance portability, and improve comfort and rest during seated travel.

It will be appreciated by those skilled in the art that the present invention is not limited to the specific embodiments described herein. Various modifications and adaptations may be made in light of the above teachings without departing from the scope and spirit of the invention, which is defined solely by the following claims.

What is claimed is:

1. A self-storing multifunctional travel set comprising:
a) a compressible headrest pillow defining an internal storage cavity;
b) an eye mask removably attachable to the headrest pillow via a fastening mechanism;
c) a sling-style blanket comprising a flexible panel having a length and shape suitable to extend around a user's torso during use; and
d) a convertible storage configuration in which the headrest pillow encloses the eye mask and the sling-style blanket within the internal storage cavity.

2. The travel set of claim 1, wherein the set is configurable between:
a) a deployed configuration in which the headrest pillow, eye mask, and sling-style blanket are extended for individual and combined use; and
b) a stored configuration in which the eye mask and sling-style blanket are contained within the internal storage cavity of the headrest pillow.

3. The travel set of claim 1, wherein the sling-style blanket comprises at least one anchoring element adapted to engage a seat structure.

4. The travel set of claim 3, wherein the sling-style blanket is formed from a breathable material and includes one or more adjustable connectors adapted to engage a seatback.

5. The travel set of claim 1, wherein the eye mask comprises at least one elastic or adjustable strap dimensioned to extend around a user's head, the eye mask being dimensionally compactable to fit within the internal storage cavity of the headrest pillow.

6. The travel set of claim 1, wherein the internal storage cavity of the headrest pillow includes a dual-zipper closure extending along an access seam to facilitate insertion and removal of the eye mask and the sling-style blanket.

7. The travel set of claim 1, further comprising a seat attachment mechanism fixed to the headrest pillow, the seat attachment mechanism comprising one or more straps dimensioned to maintain the position of the headrest pillow relative to a seatback.

8. The travel set of claim 1, wherein the headrest pillow, eye mask, and sling-style blanket are fabricated from washable, compressible materials and are collectively dimensioned to fit within a personal travel item.

9. The travel set of claim 1, further comprising a hook affixed to the headrest pillow, the hook being physically structured to engage an external surface of a personal travel item for portable attachment.
